(19) United States
(12) Patent Application Publication
(10) Pub. No.: US 2012/0009289 A1
(43) Pub. Date: Jan. 12, 2012

(54) WATER-SOLUBLE ANTIVIRAL PRODUCT CONTAINING CYPERUS ARTICULATUS, FOR THE TREATMENT AND PREVENTION OF ACQUIRED IMMUNODEFICIENCY SYNDROME (AIDS) AND THE VARIANTS THEREOF

(76) Inventor: Erick Vidjin' Agnih Gbodossou, Lanuejois (FR)

(21) Appl. No.: 12/997,502

(22) PCT Filed: Mar. 31, 2009
(86) PCT No.: PCT/IB09/51341

§ 371 Date: Apr. 4, 2011

(51) Int. Cl. A61K 36/8905; A61P 31/18

(52) U.S. Cl. 424/774

(57) ABSTRACT

The invention relates to an antiviral product based on Cyperus Articulatus, which is virolytic, virostatic and water-soluble, for the treatment and prevention of Acquired Immunodeficiency Syndrome (AIDS) and variants thereof.

It is produced as follows:
The Cyperus Articulatus leaf after collection is dried naturally and reduced to a fine powder.
The dried powder is macerated in water sterilized with 100% methanol.
The solution is then passed through a filter with a diameter of 0.45 micron and produces a sterilized antiviral, virolytic, virostatic and water-soluble product.

The different experiments show that the product:
has an antiviral activity,
has toxic and destructive effects on cells infected by the AIDS virus,
inactivates the virus and appears to be virostatic,
is water-soluble,
is not complex and has molecules less than 10,000 Da in size and shows resistance to the protease treatment.

The invention relates to an antiviral product based on the leaf of the Cyperus Articulatus plant, which is water-soluble, virolytic and virostatic. It is intended for the treatment and prevention of Acquired Immunodeficiency Syndrome (AIDS) and variants thereof. AIDS is an infectious disease that has affected the entire planet and especially African countries. It has been noted that AIDS is often accompanied by lymphadenopathic syndromes, which, in their final stage, involve a break-down of the patient's immune defenses. The agent responsible for this pandemic was isolated in 1983 by Professor Luc Montagnier from the Institut Pasteur.

The prior art known by the inventor enabled the following documents to be identified:

The OAPI patent no. 09043, granted to Joseph Cruz on Mar. 31, 1991, relating to new antiviral compositions based on Mimosa Pudica Linn leaf extract. OAPI patent no. 11602, granted to Dr. Erick Gbodossou, relating to a composition of extracts of five plants (Momordica Balsamina, Aframomum Melegueta, Cyperus Articulatus, Ficus Iteophylla, and Tamarindus Indica). In his treatment, the absorbed product removes the AIDS virus from the stool and urine. Other documents include patent WO 94/18993 granted in September 1994 to Pharmakon USA, Inc, U.S. Pat. No. 5,484,889 granted to New York University on Apr. 16, 1992, patent WO 00 35466 granted to Paya Biotechnic Inc. on Jun. 22, 200, and U.S. Pat. No. 5,886,029 granted to Kirpal S. Dhaliwal on Mar. 23, 1999. All research currently being conducted on the AIDS virus has not yet resulted in the development of a vaccine, or in killing the virus inside the body. The current antiretrovirals mutate the pathogenic agent into increasingly virulent viruses. For the more than twenty years that the pandemic has existed, research institutes, governmental and non-governmental organizations, United Nations agencies, in short the entire scientific, political, cultural and civil society communities have been invested in the fight against HIV/AIDS in order to find a vaccine, an antiviral, often in conjunction with the World Health Organization or other partners and suppliers of funds such as the Ford Foundation.

The conventional approach to HIV treatments by using antiviral substances such as azidothymidine (AZT), DDC, 3TC, AZT/3C and indinavir enables the blood plasma viral load to be reduced to the lowest level for as long as possible. The results, even if they are positive, remain limited. On average, 50% of patients see their blood plasma viral loads fall to 50 copies/ml after a treatment of around 12 months. However, in the current state of research, problems not yet solved by the use of antiretrovirals persist, in particular the impossibility of completely eradicating the pathogen.

Immune restoration is insignificant even when the CD4 lymphocyte level has increased substantially, as the persistent toxicity of antiretrovirals has not yet been resolved.

Other problems also have not been solved by the use of antiretrovirals, such as the risk of early development of antiretroviral resistance, and the uncertainty regarding the duration of efficacy of the treatments. Constraints associated with the use of the current antiretrovirals should also be noted, because patients are required to ingest some twenty products each day, and the cost is very high with respect to the purchasing power of the African populations, who are the most widely exposed to the HIV/AIDS infection. Moreover, with regard to current technological progress, there is still no water-soluble injectable antiretroviral product. No product of this type is known at present. One of the recommendations for prevention is the use of a condom; it is known that condom use not only limits procreation, but it is known in particular not to be entirely effective. The international community is becoming increasingly aware of the need to involve healers in the prevention of HIV and to call upon traditional medicine. The product of the invention is based on leaves of the Cyperus Articulatus plant, which is water-soluble, virolytic and virostatic. Studies on this plant have been conducted by the Scientific Research Department of Senegal relating to its botanical, pharmacological and chemical aspects, and analyses of the plant extract have been conducted by the laboratory of the Morehouse School of Medicine (Atlanta, Ga., USA) on the basis of a reference system developed by the inventor.

### I—Botanical, Pharmacological and Chemical Study

### Name of the Plant: Cyperus Articulatus

### Botanical Aspect

Perennial seed grass, rhizomatous, straight, cylindrical stems reaching a height of 1.75 meters and a diameter of 8 millimeters. Leaves reduced to seeds against the stem. Flower: umbrellas with irregular peduncles.

### Chemical Aspect

The chemical analysis yields sesquiterpenes and sesquiterpene alcohols with a mild odor. Cyperenone in the form of dinitro-2,4 phenylhydrazone was detected and named isopatchoulene-4. It enables the HIV virus to be removed from various infected tissues by promoting its elimination through urine and feces.

### Pharmacological Aspect

The leaf has a medical action. The aromatic properties of the Cyperus Articulatus leaf cause a sensation of diffuse heat throughout the body, and the plant acts as a sedative in dyspeptic disorders.

### II—In Vitro Study of Cyperus Articulatus

The leaf, dried and reduced to powder, was given to the Virology team at the Morehouse School of Medicine (Atlanta, Ga., USA) in order to verify its in vitro activity on the virus and variants thereof, on the basis of the reference system of the inventor.

### A—Objectives

This involved determining whether the Cyperus Articulatus extracts block the replication of the AIDS virus and whether the dried Cyperus Articulatus powder can be water-soluble. The research also aims to study the direct effect of the product on cells infected by the Acquired Immunodeficiency Syndrome (AIDS) virus and variants thereof.

### B—Implementation of Tests

The soluble fractions of the extracts of the leaf mixed in 1 mg of p24 equivalent of the virus (NL4-3KFS). The virus and the extract are initially tested from 10 mg for 1 microgram of extract. The extracts must be tested for a current application and demonstrate a direct effect on the virus. This may include the blocking of the protein of the envelope of the virus gp120 or the dissolution of lipids contained in the envelope of the virus.

Alternatively, it is possible that the active ingredients may integrate virions and affect the viral capsids or the catalytic proteins such as reverse transcription, protease or integrase.

After a preliminary incubation of 15 min with the extract, it is necessary to add the extract/virus mixture to tissue cultures from which the support was taken. The virus must absorb it for 30 min at a temperature of 37° C. The culture is then washed three times with PBS and finally preserved in a cool medium. For the initial studies, it is necessary to use cells of the MAGI indicator, which are HeLa cells designed to be infected by HIV-1. After infection, these cells produce beta-galactosidase, which can be detected as a blue color at the development of the infection. A comparison of the number of treated blue cells with untreated cells provides a relative indication of the inhibition. The virus used in the initial studies must not have any inhibition, and is therefore limited to a single step of infection of the cell. This single cell infection step is far from being the most highly indicated approach for determining the effects related to the infection. It is important in these tests to establish an appropriate dose that is not toxic to the cells and that still contains a maximum concentration of soluble substance. If an antiretroviral activity is found in the initial test, it must be determined which part of the viral life cycle is being affected. The specific viral isolators and the “indicator” cells can be used to determine the relative performance of the virus entry. If the virus entry is intact, it is possible to determine the degree to which the reverse transcription has been affected by using PCR tests, which detect various steps in the formation of proviral DNA. The capacity for producing intact virions may be determined directly by using standard p24 ELISA tests while the viral maturation can be determined by viral proteins of masses present in the virions. It is also necessary to be capable of at least making an initial determination of the nature of the soluble substance. The protease extract treatment makes it possible to determine whether the proteins present in the extract are responsible for the activity.

On the basis of the objectives and the directives, experiments have been conducted.

### III—Experiments on the Characterization of the Product

A—Solubility of the Cyperus Articulatus Extract

1 gram of dried Cyperus Articulatus powder is macerated in distilled water sterilized with 100% methanol and left to rest for one hour at room temperature. The solution obtained is then passed through a sterilization apparatus with a 0.45-micron filter in order to retain the remaining insoluble material.

The soluble fraction is free of bacteria. The substances present at the beginning in the dry matter are completely dissolved. The solution obtained is a maximum concentration of the dried powder of around 100 mg/ml.

### B—Effect of the Product on Infected Cells

To determine whether the soluble extract has any antiviral activity, we tested its effect by using the classic MAGI cell on infected cells.
The MAGI cells are genetically modified by HeLa cells, which contain a reporter gene cassette including viral LTR, placed upstream of the E. coli β-gal coding gene.
The expression of the gene is dependent on the activation by the HIV protein Tat, which requires infection by the HIV.
The infected cells turn blue and can be counted under a microscope.
We inoculated two plates with 1 ng of the HIV KFS virus.
At the same time, 200 μl of extract are added to the cells and placed in incubation for two hours.
The cells are washed twice and the extract is returned to a cool medium. The cells infected for 48 hours disappear in the presence of the product extract.

In view of this observation, it can be confirmed that the extract acts directly on the virus and that predominantly affects infected target cells. To do this, we performed the following test.

We created two additional plates containing non-infected living cells. One of the plates is a control. The antiviral product based on Cyperus Articulatus is introduced in the second plate. 30 minutes later, the AIDS virus is injected into the same plate containing the MAGI test cells before being returned to the incubation temperature. These living cells did not turn blue, i.e. they were not infected.

This experiment allowed us to conclude that:
The product has an antiviral activity.
The product inactivates the virus.
The product destroys cells infected by the Acquired Immunodeficiency Syndrome (AIDS) virus and variants thereof.

### C—SELDI Mass Spectrometry of the Mixed Product

The product after study showed that the active principles are not complex and the molecules located there are less than 10,000 Da in size and demonstrate resistance to the protease treatment.

### IV—Results

These different experiments show that the product:
has an antiviral activity,
has toxic and destructive effects on cells infected by the AIDS virus,
inactivates the virus and appears to be virostatic,
is water-soluble,
is not complex and has molecules less than 10,000 Da in size and shows resistance to the protease treatment.

The antiviral product based on Cyperus Articulatus for the treatment and prevention of Acquired Immunodeficiency Syndrome (AIDS) and variants thereof, of the invention, is produced as follows.

The Cyperus Articulatus leaf after being collected is dried naturally and reduced to a fine powder. The dried powder is macerated in water sterilized with 100% methanol. The solution is then passed through a filter with a diameter of 45 microns and produces an antiviral, virolytic, virostatic and water-soluble product.

The product can be used as a vaginal microbicide, but also in any other form.

It has been demonstrated in in vitro tests that the solution obtained blocks the development of the Acquired Immunodeficiency Syndrome (AIDS) virus and variants thereof, and indirectly stimulates and increases the reticuloendothelial system, thereby enhancing the patient's immune defenses.

1. An antiviral product based on Cyperus Articulatus for therapeutic use in treatment and prevention of Acquired Immunodeficiency Syndrome (AIDS) and variants thereof.

2-10. (canceled)

11. The product of claim 1 further comprising:
a dried Cyperus Articulatus leaf reduced to a fine powder, wherein the powder of the Cyperus Articulatus leaf is macerated in water sterilized with 100% methanol to obtain a solution passed through a 0.45-micron filter.

12. The product of claim 11, wherein the product is water-soluble.

13. The product of claim 11, wherein the product has an antiviral activity.

14. The product of claim 11, wherein the product has toxic and destructive effects on cells infected by the Acquired Immunodeficiency Syndrome (AIDS) and variants thereof.

15. The product of claim 11, wherein the product inactivates the virus and is virostatic.

16. The product of claim 11, wherein the product is not complex and has molecules less than 10,000 Da in size and shows resistance to protease treatment.

17. The product of claim 1, wherein the product stimulates a reticuloendothelial system.

18. The product of claims 1, wherein the product can be used as a vaginal microbicide and in any other form.

19. The product of claim 11, wherein the product stimulates a reticuloendothelial system.

20. The product of claim 11, wherein the product can be used as a vaginal microbicide and in any other form.

21. The product of claim 1, wherein a concentration and a purification of the Cyperus Articulatus leaf extract are performed according to conventional methods used in chemistry and biochemistry.

22. The product of claim 11, wherein a concentration and a purification of the Cyperus Articulatus leaf extract are performed according to conventional methods used in chemistry and biochemistry.

23. A therapeutic use of the product of claim 11 in treatment and prevention of Acquired Immunodeficiency Syndrome (AIDS) and variants thereof.

24. The therapeutic use of claim 23 wherein the product stimulates a reticuloendothelial system.

25. The therapeutic use of claim 23 as a vaginal microbicide.

26. A method of preparation of an antiviral product based on Cyperus Articulatus and for therapeutic use in treatment and prevention of Acquired Immunodeficiency Syndrome (AIDS) and variants thereof.

27. The method of claim 26 further comprising the steps of:
a) a first step for collecting a dried Cyperus Articulatus leaf reduced to a fine powder,
b) a second step for macerating the powder of the Cyperus Articulatus leaf in water sterilized with 100% methanol to obtain a solution,
c) a third step for passage of the solution through a 0.45-micron filter.

28. The method of claim 27 wherein the method comprises steps for concentration and purification of the Cyperus Articulatus leaf extract which are performed according to conventional methods used in chemistry and biochemistry.
